SEQUENCE LISTING (7 sequences)

SEQ ID NO: 1 - DNA, 25 bp, Artificial Sequence
  PCR Primer
ctgagggaggaaaatcgcttgaact

SEQ ID NO: 2 - DNA, 20 bp, Artificial Sequence
  PCR Primer
ctctgaatctcccgcaatgt

SEQ ID NO: 3 - DNA, 19 bp, Artificial Sequence
  PCR Primer
gggaaagaaaacatcaagg

SEQ ID NO: 4 - DNA, 22 bp, Artificial Sequence
  PCR Primer
aggttacaagacaggtttaagg

SEQ ID NO: 5 - DNA, 21 bp, Artificial Sequence
  PCR Primer
agtctgttgtgagcccttcca

SEQ ID NO: 6 - DNA, 21 bp, Artificial Sequence
  PCR Primer
cgacgacaacaccaattttcc

SEQ ID NO: 7 - DNA, 30 bp, Artificial Sequence
  PCR Primer
aagttttgtttagaggagaacgagcgccct

(19) United States
(12) Patent Application Publication
(10) Pub. No.: US 2008/0020382 A1
(43) Pub. Date: Jan. 24, 2008

(54) Biological Microbeads for Various Flow Cytometric Applications

(75) Inventors: Gabor Szabo, Debrecen (HU); Gyorgy Lustyik, Pecs (HU); Miklos Szabo, God (HU)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 11/575,859

(22) PCT Filed: Oct. 7, 2005
(86) PCT No.: PCT/US05/36038

§ 371 Date: Mar. 22, 2007

Related U.S. Application Data
(60) Provisional application No. 60/616,997, filed on Oct. 8, 2004.

(51) Int. Cl. G01N 33/554; C12Q 1/68; G01N 33/569; G01N 33/53

(52) U.S. Cl. 435/6; 435/7.1; 435/7.31; 435/7.32; 435/7.33

(57) ABSTRACT

The invention relates to biological microbeads, and in certain embodiments, to the use thereof in flow-cytometric applications. Biological microbeads are fixed cells that may be surface-modified with proteins or other molecules that have specific binding properties. These surface-bound proteins may bind to target compounds bearing fluorescent labels that facilitate detection by flow cytometry. Provided are compositions and methods for the use of biological microbeads as an inexpensive alternative to synthetic microbeads for in an extensive number of flow-cytometric applications, including quantitative PCR, detection of nucleases, detection of proteases, and immunoassays.

### FIELD OF THE INVENTION

The invention relates to compositions and methods involving biological microbeads, which are particularly useful in connection with flow-cytometric applications.

### BACKGROUND

The development and availability of microbeads amenable to flow-cytometric analysis has opened a new chapter in the field, making quantitative measurement of molecules in a homogeneous phase possible. Since the spectrum of molecular entities analyzable this way encompass proteins (Frengen J. et al. (1993) Clin Chem 39:2174-81), nucleic acids (Spiro A. et al., (2000) Appl Environ Microbiol 66:4258-65, Spiro A. et al., (2002) Appl Environ Microbiol 68:1010-3) and molecules recognized by these (Iannone M. A. et al., (2001) Cytometry 44:326-37., Vignali D. A., (2000) J Immunol Methods 243:243-55, Kellar K. L. et al., (2002) Exp Hematol 30:1227-37), the microbead-based flow-cytometric technology can provide a universal measuring platform for many laboratory purposes. Titration of proteins by microbead-based flow-cytometric immunoassays have been demonstrated for several proteins and proved to be viable alternatives to conventional technologies, like ELISAs (Pickering J. W. et al., (2002) Clin Diagn Lab Immunol 9:872-6., Dasso J. et al., (2002) J Immunol Methods 263:23-33).

The fields of application include techniques serving microbiological purposes, such as immunoassays of bacterial (Park M. K. et al., (2000) Clin Diagn Lab Immunol 7:486-9) or viral (Yan X. et al., (2004) J Immunol Methods 284:27-38) antigens, or analysis of antiviral/antibacterial antibodies (Pickering J. W. et al., (2002) Clin Diagn Lab Immunol 9:872-6., Martins T. B., (2002) Clin Diagn Lab Immunol 9:41-5). While this approach matches conventional methods in sensitivity, reproducibility and simplicity, it seems to have significant advantages by virtue of the possibility for multiplex analysis. Furthermore, sequence-specific capture of PCR-amplified genomic or cDNA sequences allows detection of single nucleotide polymorphisms (SNPs) (Taylor, J. D. et al., (2001) Biotechniques 30:661-699, Ye, F. et al., (2001) Hum Mutat 17:305-16., Rao, K. V. et al. (2003) Nucleic Acids Res 31:e66.), and also turns this platform into a possible alternative to microarrays on chips to be used for the characterization of gene expression profiles (Brenner S et al., (2000) Proc Natl Acad Sci USA; 97(4): 1665-70.).

The realm of possibilities is virtually unlimited; most of the routine techniques of a biochemical laboratory can be adapted to flow-cytometric methodology. However, the current methods rely on commercially-supplied synthetic or polymeric microbeads which may be expensive and/or have limited binding capacity. In view of these observations, there is a need in the art for alternatives to conventional, commercially-available polymeric microbeads that can provide extremely flexible, readily available and inexpensive microbead systems that are compatible with ordinary flow-cytometric instrumentation.

### SUMMARY OF THE INVENTION

The invention disclosed herein relates to the use of biological microbeads to bind target compounds that can subsequently be analyzed by methods such as flow cytometry.

Embodiments of the present invention relate to methods involving binding biological microbeads to a target compound, comprising providing a composition comprising a target compound, and contacting the composition with a quantity of biological microbeads sufficient to bind at least a portion of the target compound to produce a quantity of biological microbead-bound target compounds.

Further embodiments provide methods wherein the biological microbeads comprise proteins that are covalently attached to fixed cells.

Still further embodiments of the invention provide for methods wherein the fixed cells are selected from the group consisting of bacterial cells, yeast cells, and combinations thereof, and additionally, methods wherein the bacterial cells comprise Staphylococcus aureus cells, or the yeast cells comprise the strain ND6.

Other embodiments of the invention pertain to methods wherein the proteins are selected from the group consisting of avidin, streptavidin, and combinations thereof.

Additional embodiments of the invention relate to methods wherein the target compound comprises a compound selected from the group consisting of proteins, nucleic acids, antibodies, and combinations thereof.

Further embodiments of the invention relate to methods wherein the target compound comprises biotin.

Still further embodiments of the invention relate to methods wherein the biological microbead-bound target compounds are detected by flow cytometry.

Embodiments of the invention relate to compositions comprising cells that have been fixed and cross-linked to a protein, wherein the protein is adapted to bind a target compound.

Further embodiments of the invention relate to compositions wherein the fixed cells are selected from the group consisting of bacterial cells, yeast cells, and combinations thereof, as well as compositions wherein the bacterial cells comprise Staphylococcus aureus, and the yeast cells comprise strain ND6.

Still further embodiments of the invention relate to compositions wherein the protein is selected from the group consisting of avidin, streptavidin, and combinations thereof.

Other embodiments of the invention relate to compositions wherein the target compound comprises a compound selected from the group consisting of proteins, nucleic acids, antibodies, and combinations thereof.

Additional embodiments of the invention pertain to compositions wherein the target compound comprises biotin.

Embodiments of the invention relate to methods of detecting target compounds, comprising providing a composition comprising a target compound, contacting the composition with a quantity of biological microbeads sufficient to bind at least a portion of the target compound to produce a quantity of biological microbead-bound target compound and a quantity of unbound target compound, separating the biological microbead-bound target compound from the unbound target compound, and detecting the biological microbead-bound target compound.

### BRIEF DESCRIPTION OF THE DRAWINGS

FIG. 1a depicts a confocal microscopic picture of a fixed and avidinated yeast cell labeled with biotinylated and 6FAM-labeled PCR products at high magnification (FIG. 1a), in accordance with an embodiment of the present invention.

FIG. 1b depicts confocal microscopic pictures of fixed and avidinated yeast cells labeled with biotinylated and 6FAM-labeled PCR products at low magnification (FIG. 1b) in accordance with an embodiment of the present invention.

FIG. 2a shows a saturation curve for the binding of a biotinylated and 6FAM-labeled PCR product to biological in accordance with an embodiment of the present invention. The non-specific binding, which is shown in the dotted line, is the titration curve obtained with fluorescent but unbiotinylated ligands.

FIG. 2b shows a saturation curve for the binding of a biotinylated and 6FAM-labeled PCR product to biological microbeads in accordance with an embodiment of the present invention. The non-specific binding, which is shown in the dotted line, is the titration curve obtained with fluorescent but unbiotinylated ligands.

FIG. 2c shows a saturation curve for the binding of biotinylated and FITC-labeled casein to biological microbeads, in accordance with embodiments of the present invention. The non-specific binding, which is shown in the dotted line, is the titration curve obtained with fluorescent but unbiotinylated ligands.

FIG. 2d shows a saturation curve for the binding of a biotinylated and 6FAM-labeled PCR product to commercial microbeads in accordance with an embodiment of the present invention. The non-specific binding, which is shown in the dotted line, is the titration curve obtained with fluorescent but unbiotinylated ligands.

FIG. 3 shows a copy number determination that is linear between ˜1×10²-5×10¹⁰ molecules of the template at 25 cycles in accordance with an embodiment of the present invention.

FIG. 4a shows the amount of PCR product formed as a function of the number of amplification cycles, using 3.21×10¹¹ (continuous line) or 6.42×10¹¹ (dotted line) copies of MLL plasmid template, in accordance with an embodiment of the present invention.

FIG. 4b shows a titration of the MLL plasmid template copy number in PCR reactions performed using biotinylated and 6FAM-labeled primers in accordance with an embodiment of the present invention.

FIG. 5a shows a determination of Xba I restriction enzyme activity in accordance with an embodiment of the present invention.

FIG. 5b shows a determination of Pvu II restriction enzyme activity in accordance with an embodiment of the present invention.

FIG. 6a shows a titration of proteinase K concentration on microbeads in accordance with an embodiment of the present invention.

FIG. 6b,c shows the results of a fluctuation analysis ranked in the order of average fluorescence intensities in accordance with an embodiment of the present invention, with the observed data compared to the values calculated based on a Poisson distribution of lambda=1.

FIG. 7 depicts a method of using biological microbeads for immunoassays in accordance with an embodiment of the present invention.

FIG. 8a shows a titration of a dilution series of AFP in accordance with an embodiment of the present invention.

FIG. 8b shows titration of a dilution series of βhCG in accordance with an embodiment of the present invention.

FIG. 8c shows a forward-scatter/forward light scattering dot-plot accordance with an embodiment of the present invention.

FIG. 8d shows a forward light-scattering distribution histogram of a mixture of five yeast cell samples stained with a tenfold dilution series of 1 mg/ml fluorescein isothiocyanate (background fluorescence: Bgr).

### DESCRIPTION OF THE INVENTION

The invention relates to biological microbeads and their use in connection with flow-cytometric applications. Certain components of the invention are discussed in a publication by Pataki et al., which is incorporated herein by reference in its entirety (Pataki, J. et al., (2005) Cytometry Are-publication Sep. 14, 2005). The biological microbeads of the present invention include fixed prokaryotic or eukaryotic cells (e.g., bacteria, yeast, etc.) to which the proteins avidin, streptavidin, or any of their related molecules are covalently or noncovalently immobilized. Alternatively, prokaryotic or eukaryotic cells that exhibit avidin, streptavidin, or related proteins on their surface due to the expression of genes that specify such proteins are considered to be within the scope of the invention, as proteins that are expressed on the surface of a cell are generally also covalently or noncovalently attached to said cell.

These biological microbeads can be used in the same fashion as conventional polymeric or synthetic microbeads; for instance, those that are composed of polystyrene, carboxyl-styrene, or carboxylated microspheres. See, e.g., Krupa et al., “Quantitative bead assay for hyaluronidase and heparinase I,” 319 Analytical Biochemistry 280-286 (2003); Yan et al., “Microsphere-based duplexed immunoassay for influenza virus typing by flow cytometry,” 284 J. Immunological Methods 27-38 (2004); Xu et al., “Multiplexed SNP genotyping using the Qbead system: a quantum dot-encoded microsphere-based assay,” Nucleic Acids Research, vol. 31, no. 8 (2003); and Kellar & Douglass, “Multiplexed microsphere-based flow cytometric immunoassays for human cytokines,” 279 J. Immunological Methods 277-285 (2003). All references listed herein are incorporated by reference as though fully set forth.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992); and Sambrook and Russell, Molecular Cloning: A Laboratory Manual 3rd ed., Cold Spring Harbor Laboratory Press (Cold Spring Harbor, N.Y. 2001) provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

Flow cytometry is a technique in which microscopic particles are suspended in a stream of fluid, and are measured or quantitated by a laser beam based on chemical or physical characteristics of the particle, such as fluorescence or light scattering. A number of different types of particles may be analyzed by flow cytometer, including live cells, fixed cells, and synthetic (or polymeric) microbeads, and biological microbeads. Flow cytometers are capable of measuring features of particles that have been labeled with compounds that make them fluoresce. Flow cytometry enables researchers to observe characteristics of a large number of particles, one particle at a time.

As used herein, the term “target compound” refers to compounds that bind to molecules that are affixed to biological microbeads. Examples of target compounds include but are not limited to nucleic acids, proteins, antibodies, sugars, and small molecules.

In various applications of the invention, antibodies can be covalently attached to the biological microbeads for immunoassay-type studies. Alternatively, polymerase chain reaction (“PCR”) products may be prepared using biotinylated and fluorescent dye-labeled primers on the two ends. Furthermore, the biological microbeads may be used for the purposes of, for instance, quantitative PCR, the detection of nucleases, the detection of proteases, the detection of genetic mutations (e.g., insertions, deletions, mutations, single nucleotide polymorphisms (“SNPs”), rearrangement), and any other applications where polymeric microbeads are generally applied. Any of these methodologies may be applied alone (i.e., for titration of a single molecule) or, in part because they can be easily addressed by fluorescent dyes, in a multiplex format (i.e., using a series of microbeads resolved side-by-side in a flow cytometer) much like commercial microbeads (e.g., (strept)avidinated microbeads produced by Sigma, Becton Dickinson, etc.). Triggering flow cytometric detection on a dot-plot, using a single color and one of the scatter signals, for example, several (i.e., at least six) microbead populations can be resolved side-by-side, which extends to 36 (using two colors) or more, using cells of different size (e.g., yeast plus bacteria).

Biological microbeads are similar in functionality to the conventionally-used synthetic or polymeric microbeads, but rather than comprising a synthetic compound, biological microbeads comprise cells that have been fixed. Methods for fixing cells are well known in the art. In general, a number of different types of cells, including bacterial and yeast cells, are suitable for production of biological microbeads and subsequent use in flow cytometric applications.

The biological microbeads of the present invention make measurements relatively inexpensive, and their binding capacity is believed to exceed that of polymeric and synthetic commercial beads that are currently available. In fact, the biological microbeads of the present invention exhibit a 10-30-fold ratio of specific over non-specific binding (measured using nonbiotinylated ligands), depending upon the ligand used; thereby allowing accurate and comfortable titrations. The versatility of the techniques made possible using biological microbeads, encompassing a broad range of biochemical and molecular biological methods, makes the use of biological microbeads viable alternatives or supplements to research and diagnostic applications; particularly those applications that would otherwise involve the use of polymeric or synthetic microbeads.

### EXAMPLES

The following examples illustrate the use of the biological microbeads of the present invention in connection with a variety of techniques and protocols. These Examples are included for purposes of illustration only, and are not intended to limit the scope of the range of techniques and protocols in which the biological microbeads of the present invention may find utility, as will be appreciated by one of skill in the art and can be readily implemented.

### Example 1

### Preparation and Evaluation of Biological Microbeads

A strain of Staphylococcus aureus (buffered aqueous suspension of formalin-fixed protein A-negative bacteria) was purchased from Sigma. The ND6 strain of Saccharomyces cerevisiae used to demonstrate the principle, was a gift from I. D. Hickson (Oxford, UK). The cells were fixed at 4° C., overnight, in 2% paraformaldehyde (PFA) solution prepared freshly in PBS. Avidin conjugates were produced by carbodiimide coupling (Hermanson G T., (1996) Bioconjugate techniques. San Diego, London: Academic Press, pages 170-173); as described below). The avidin-labeled biological microbeads were stored in PBS containing sodium azide (0.02%) at 4° C., and were found stable for at least a year.

For conjugation of yeast cells or bacteria with avidin or streptavidin, 10⁸ fixed yeast cells or bacteria were transferred to a 2 ml conical centrifuge tube, washed 3× in PBS by centrifugation and resuspended in 200 μl of PBS. In a separate tube, 20 mg avidin (from Inovatech Europe B.V., Zeewolde, The Netherlands) or streptavidin (Roche Diagnostics), and 32 mg EDC (1-ethyl-3-(3-dimethylaminopropyl)carbodiimide, Sigma) were dissolved in 100 μl PBS. The avidin/streptavidin-EDC solution was added to the cells, and these samples were incubated amid constant shaking on a mixing device, at room temperature, for 18 to 24 hours. The avidin/streptavidin conjugated biological microbeads were washed 5× in 1 ml PBS and stored at 4° C. after adding NaN₃ to 0.02%.

To measure binding of ligands to the beads, biotinylated and fluorescent (in control samples just fluorescein-labeled) casein or PCR products were added to 10,000 biological microbeads or, for comparison, to 10,000 polymeric beads (6 μm diameter, streptavidin-coated, plain beads purchased from Polyscience AG, Switzerland) in 50 μl PBS, and incubated at RT for 40 mins, and washed twice by centrifugation, Polymeric beads and yeast cells were centrifuged at 1000 g, bacteria at 2000 g, for 10 mins.

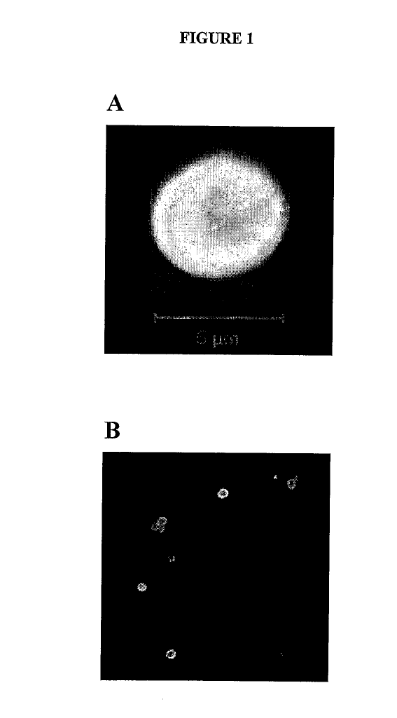

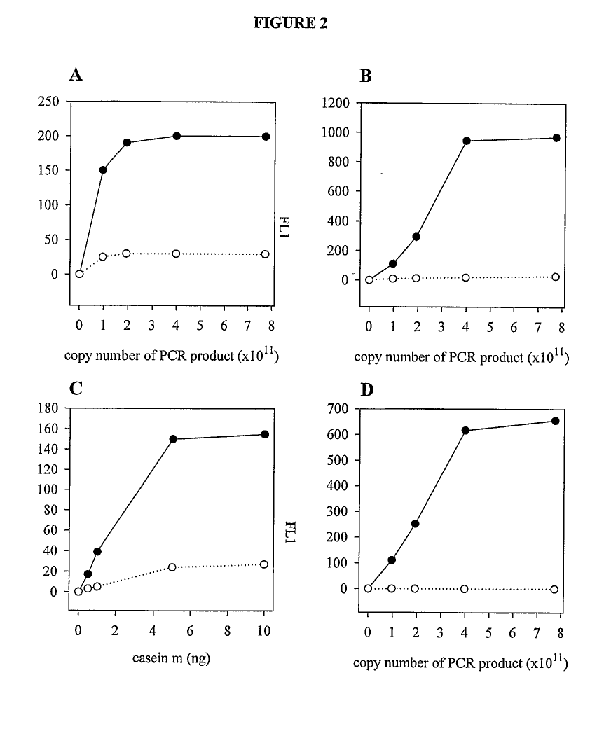

As FIG. 1a and FIG. 1b demonstrate, the avidinated yeast cells bind biotinylated nucleic acids (and proteins; not shown) mainly on their surface, and this binding is in great part specific, as revealed by the low level of staining with non-biotinylated ligands (see FIGS. 2a through 2d). The level of nonspecific binding varied between batches of avidinated yeast samples; see FIG. 2b as an example of a very low degree of nonspecific binding, comparable to that of commercial, polymeric beads (FIG. 2d). The binding capacity of the avidinated yeast particles was comparable to (or slightly exceeded that of) the commercial beads (compare FIG. 2b and FIG. 2d). At the usual efficiency of avidination provided by the conjugation reaction (Hermanson G T., (1996) Bioconjugate techniques. San Diego, London: Academic Press; 170-173), a dynamic range of ˜2 logs may be achieved. It is estimated that the specific binding sites were saturated by ˜10⁷ ligands per yeast cell (based on FIG. 2b). The biological microbeads proved advantageous also because they were readily centrifuged even at high protein concentrations (data not shown).

For confocal laser scanning microscopy, pictures were taken by a Zeiss LSM 510 instrument. The 488 nm line of an Argon-ion laser was used for the excitation of fluorescein. The 2% PFA-fixed microbead samples were dissolved in Prolong antifade (from Molecular Probes, Oregon, USA) and deposited on slides.

### Example 2

### Quantitative PCR Using Biological Microbeads

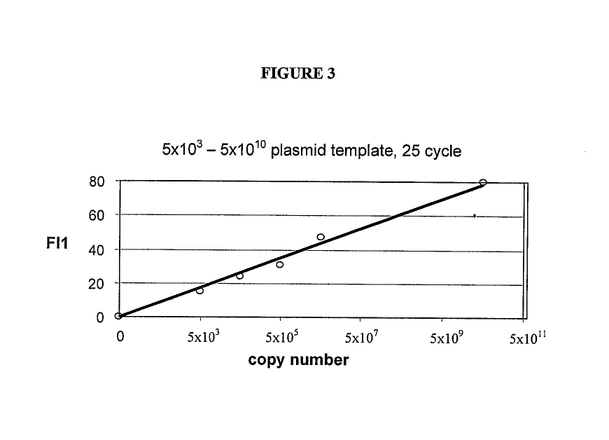

FIG. 2 depicts saturation curves, using avidinated yeasts and either a 6FAM/biotin-labeled PCR product (spec) or a PCR product labeled only with 6FAM (aspec). The copy number of the template can be quantitated in PCR reactions measuring the fluorescence of the bead-immobilized PCR products, prepared as described above. A single time point, between 5-25 cycles, depending upon the template concentration, is compared to a calibration curve. At 25 cycles, as shown in FIG. 3, the copy number determination is linear between 100-1,000 molecules of the template.

PCR reactions were performed in 50 μl volume of 1× reaction buffer containing 2.5 mM dNTP-solution (from Promega Biosciences, Madison, USA), template DNA, 0.4 μM of sense and 0.4 μM of antisense primers, 1.5 mM MgCl₂ and 2.5 U Taq polymerase (Fermentas Life Sciences, USA). Primers delimiting a 720 bp long fragment within the MLL bcr: 5′(biotin)-CTG AGG GAG GAA AAT CGC TTG AAC T-3′ (SEQ ID NO:1) and 5′-(6FAM)-CTC TGA ATC TCC CGC AAT GT-3′ (SEQ ID NO:2), were obtained from Bioscience (Washington, USA). Primers defining a 340 bp region within the human β-globin gene (2^{nd} exon) were: 5′-(Cy3)-GGGAAAGAA AACATCAAGG-3′ (SEQ ID NO:3), and 5′-(biotin)-AGGTTACAAGACAGGTTTAAGG-3′ (SEQ ID NO:4) (Merck & Co., Inc, USA).

The PCR products were analyzed on 2% agarose gels run in 1×TAE ((0.04 M TRIS, 0.02 M acetic acid, 0.01 M EDTA (pH 8.0)), purified on QIAquick PCR purification kits (Qiagen, Germany) and eluted in 50 μl sterile TE (10 mM TRIS, 1 mM EDTA, pH 8.0). Small aliquots of these samples were added to 10,000 beads in PBS, incubated, washed and analyzed by FACScan (see below). Titration of template copy number was performed at 25 cycles; the samples were diluted before addition of the beads so that the beads were never saturated by ligand.

Real time quantitative PCR (qPCR): qPCR was carried out on an ABII7900 Real Time Sequence Detection System. The oligos used were as follows:

(SEQ ID NO:5)

fw5′-3′
AGTCTGTTGTGAGCCCTTCCA,

(SEQ ID NO:6)

rev5′-3′
CGACGACAACACCAATTTTCC,
and

(SEQ ID NO:7)

probe5′-3′
Fam-AAGTTTTGTTTAGAGGAGAACGAGCGCCCT-Tamra.

The reactions were performed in a volume of 22 μl according to the manufacturer's instructions. For standard curve calibration a plasmid carrying the MLL-bcr (gift from Peter D. Aplan, NIH, Bethesda, Md.) was used. The number of copies was calculated by comparison to the standard curve.

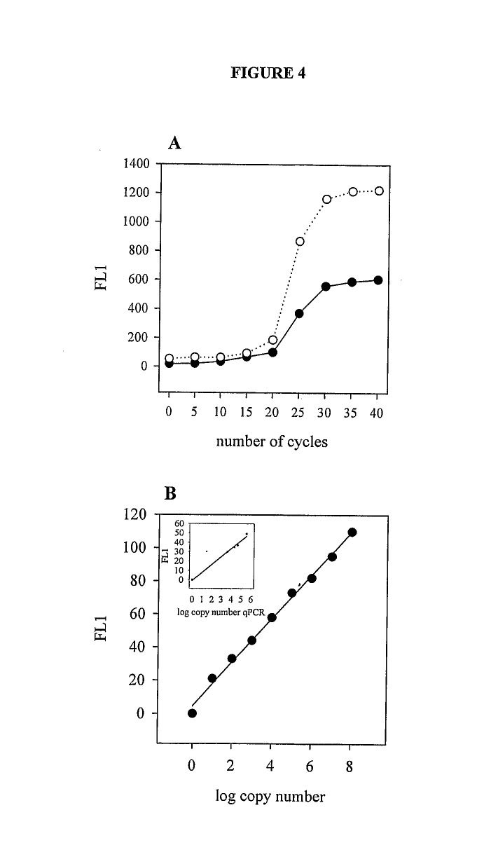

Biological microbeads can also be utilized in single-point measurements for quantitative PCR purposes, as demonstrated in FIG. 4a and FIG. 4b. In FIG. 4a, PCR reactions were run for different numbers of cycles, and were carried out in parallel. In FIG. 4b, the copy numbers were calculated from the 260 nm absorption reading of the undiluted DNA solution. The products captured on fixed/avidinated yeast cells after 25 cycles of amplification were measured by flow-cytometry (FL1). The inset shows the correlation of the qPCR-determined log copy numbers with the FL1 values, in a separate experiment.

The samples were added to fixed/avidinated yeast cells and measured by flow-cytometry (FL1). PCR products were detected at 20 cycles, long before polymerization would lose its linear relationship with template copy number. At this cycle-number, the amount of PCR products (generated using biotinylated and fluorescent primers), was proportional to the logarithm of template copy number in a wide range, between 1 and 10⁸ (FIG. 4b). The semi logarithmic relationship implies a relative loss of sensitivity toward higher concentrations of biotin- and fluorescein-labeled PCR products, perhaps due to the presence of both more and less accessible avidin molecules on the yeast cell surfaces.

Flow cytometric measurements were conducted using a Becton-Dickinson FACScan flow cytometer (Mountain View, Calif., USA). Fluorescence signals were collected in the logarithmic mode, and subsequently converted to linear units and the data were analyzed by the BDIS CELLQUEST 3.3 (Becton-Dickinson) software. Samples were run at high speed, the applied laser power was 15 mW, fluorescence signals were detected in the FL1 and FL2 channels, through the 530/30 and 585/42 interference filters of the instrument, respectively.

### Example 3

### Detection of Nucleases Using Biological Microbeads

The methodical scenario described above is readily applicable for detection of nucleases. PCR products containing a restriction enzyme recognition site were prepared using biotinylated and fluorescent primers, and were immobilized on beads and digested with the enzyme of interest. Both nonspecific endonucleases and restriction enzymes can be detected with extreme sensitivity; down to at least 10⁻⁵ units of the enzyme.

For the titration of restriction enzyme activities, labeled PCR products, with and without the appropriate recognition site, were mixed and digested with Xba I or Pvu II. The PCR product containing no recognition sites was prepared by using human genomic DNA as template, and biotinylated/Cy3-labeled primers defining a 340 bp long sequence within the β-globin gene. The 720 bp long product prepared using the MLL plasmid template contained a recognition site for both Xba I and Pvu II; this product was biotinylated and 6FAM-labeled (see primers above). 100 ng of each product was digested with aliquots of a dilution series of the enzymes (from Fermentas Life Sciences, USA), in 50 μl volume containing 1× buffer, at 37° C. for 2 hrs. After inactivation of the enzymes at 65° C. for 20 mins, 50 μl of PBS containing biological microbeads were added, the samples were further incubated for 40 mins in the dark, washed twice and analyzed in the flow-cytometer.

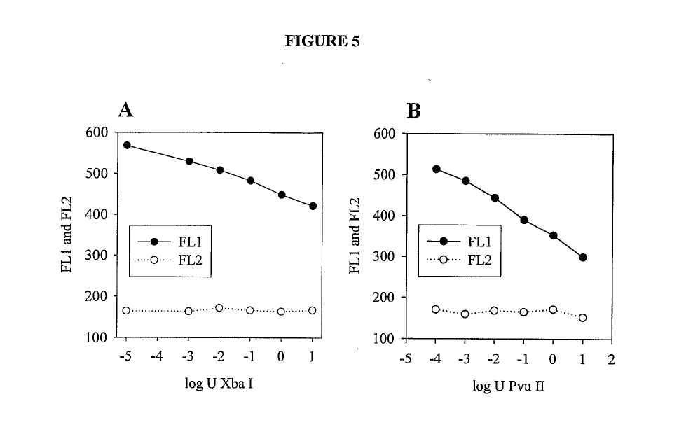

As FIG. 5a and FIG. 5b show, nuclease enzyme activities may also be readily determined by flow-cytometry, using PCR products as substrates, immobilized on biological microbeads after digestion in a homogeneous phase. A biotinylated and 6FAM-labeled PCR product containing the Xba I or Pvu II recognition sites, amplified using the MLL plasmid template has been subjected to restriction enzyme digestion by Xba I (FIG. 5a) and Pvu II (FIG. 5b). As an internal control, part of the β-globin gene that carries no such sites was amplified using human genomic DNA template, biotin- and Cy3-labeled 5′ and 3′ primers, respectively. The two PCR products were mixed at a molar concentration ratio of 1:1, immobilized on fixed/avidinated yeast cells and analyzed by flow-cytometry. FL1 shows the decrease of the MLL-related fluorescence upon digestion, while the constant values of FL2 exclude nonspecific degradation in the same sample.

Incubation with a restriction enzyme that has no recognition site in the PCR product has no effect on the average fluorescent intensity of the beads carrying the fluorescent (and biotinylated) nucleic acids (see FL2 signals). The sensitivity of such an assay exceeds that of gel electrophoretic analysis by at least ˜2 orders of magnitude. Nonspecific endonucleases, e.g. DNase I, could also be titrated using this assay, with high sensitivity, on double-stranded as well as partially single-stranded DNA molecules as substrates (data not shown).

Flow cytometric measurements were conducted using a Becton-Dickinson FACScan flow cytometer (Mountain View, Calif., USA). Fluorescence signals were collected in the logarithmic mode, and subsequently converted to linear units and the data were analyzed by the BDIS CELLQUEST 3.3 (Becton-Dickinson) software. Samples were run at high speed, the applied laser power was 15 mW, fluorescence signals were detected in the FL1 and FL2 channels, through the 530/30 and 585/42 interference filters of the instrument, respectively.

### Example 4

### Detection of Proteases Using Biological Microbeads

The method described above for the detection of nucleases is also applicable for detection of proteases. Any protease can be detected using general proteases substrates (e.g., casein, labeled with both biotin and a fluorescent dye) immobilized on beads, and specific proteases; for example, metalloproteinases can be detected using specific, bead-immobilized substrates (e.g., peptides conjugated with both biotin and a dye). In experimental procedures, the inventive method may be extremely sensitive in the case of proteinase K; a single molecule of the enzyme can be detected.

By way of example, in one embodiment of the invention, this methodology is used to detect human immunodeficiency virus (“HIV”) protease, using biotinylated+fluorescent-labeled GAG protein or the appropriate (labeled) peptide. In another embodiment of the invention, angiotensin converting enzyme (“ACE”) activity in serum is measured; a parameter of predictive significance in the case of cardiovascular diseases. Labeling of the substrate (poly)peptides can usually be random, although in certain cases particular moieties are labeled (e.g., the two ends, with biotin and the dye, respectively).

For titration of enzyme activities, Proteinase K was used to digest casein-biotin-FITC. Proteinase K (Promega Biosciences, Madison, USA) digestion of 100 ng casein-biotin-FITC was performed in 50 μl PBS/0.1% SDS (Sigma, St. Louise, Mo., USA), for 2 hrs at 37° C. After 40 mins incubation of the digests with biological microbeads at RT, in the dark, the beads were washed twice and resuspended in 500 μl PBS for flow-cytometric analysis.

For fluctuation analysis, proteinase K was serially diluted to contain 20 molecules in the complete volume, which was then divided into 20 aliquots. Each aliquot, containing a single proteinase molecule on the average, was used to digest casein-biotin-FITC as described above.

To prepare casein-biotin-FITC, case in was first biotinylated. 5 mg (20 nmole) of case in (Sigma, St. Louise, Mo., USA) was dissolved in 900 μl carbonate buffer (0.1 M NaHCO₃, pH=8.3), cleaned on Centricon YM-30 tubes (Millipore, USA Mass.), and redissolved in 900 μl carbonate buffer. 2 mg (6 μmole) of biotin N-hydroxysuccinimide-ester (Sigma, St. Louise, Mo., USA) was dissolved first in 30 μl dimethyl sulfoxide (Sigma, St. Louise, Mo., USA), then supplemented with 70 μl carbonate buffer, and the full volume (100 μl) was added to the casein solution upon vortexing. Labeling proceeded for 1 hr at RT. The protein was purified on Centricon YM-30 tubes and eluted in 500 μl carbonate buffer. Biotinylated casein was further conjugated to an F/P ratio of 1.1, with the fluorescein derivative dye 5-SFX (Molecular Probes, Oregon, USA). 1 mg of the dye was dissolved in 30 μl DMSO, and added to the casein-biotin (carbonate) solution. Following incubation at RT for 1 hr, in the dark, 1/10 volume of hydroxylamine (pH 8.5) was added to stop the conjugation reaction. After 20 mins incubation, the samples were purified on a Sephadex G-25 column and eluted in 1× PBS (150 mM NaCl, 3.3 mM KCl, 8.6 mM Na₂HPO₄, 1.69 mM KH₂PO4, pH 7.4). The casein-FITC-biotin samples were stored at 4° C., adding sodium-azide to 0.02% final concentration. To prepare casein-FITC, purified casein was directly conjugated with 5-SFX, as described above.

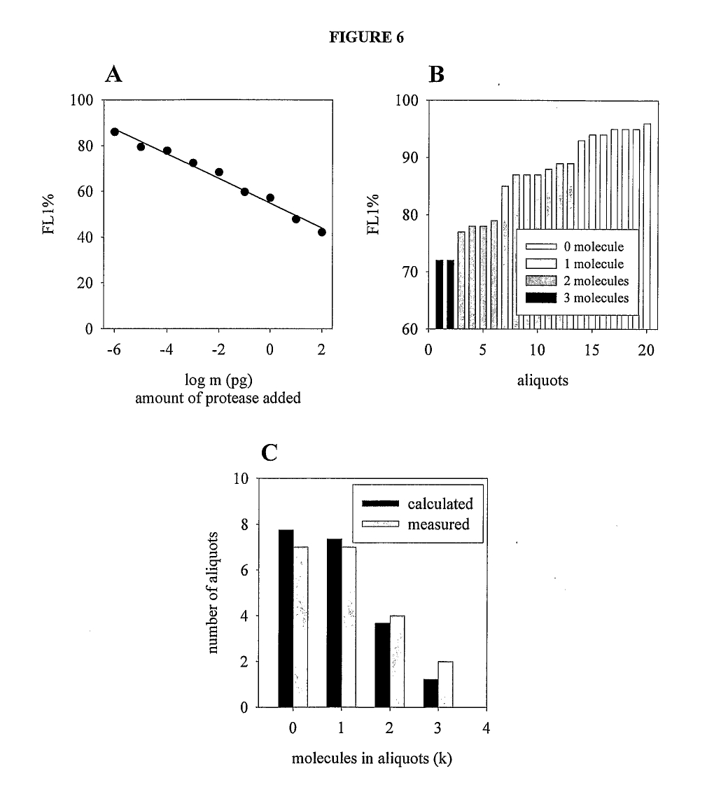

FIG. 6 demonstrates that the biological microbeads may support very sensitive assays for determining enzymatic activities for proteases. In FIG. 6a, fluorescein- and biotin-conjugated casein was digested with various concentrations of the protease, in the presence of 1% SDS; the relationship is linear to R²=0.9852. The decrease of average fluorescence (% of the initial value) is plotted against the amount of protease added (m). According to the titration curve shown in FIG. 6a, proteinase K concentrations as low as 10⁻⁶ pgs (i.e., a few molecules) can be measured in the 300 μl reaction volume.

FIGS. 6b and 6c show the results of a fluctuation analysis, when 20 aliquots of an enzyme solution diluted to contain a single enzyme molecule in each aliquot, have been compared. The average fluorescence intensities were ranked as shown in FIG. 6b (k designates the number of protease molecules assumed to be present in the aliquots). In FIG. 6c, the number of aliquots exhibiting approximately equal levels of average fluorescence after digestion were plotted at k=0, 1, 2 and 3 as measured values; the calculated values were obtained assuming a Poisson distribution of λ=1. Results of a single experiment are shown. The fluctuation analysis shown in FIG. 6b and FIG. 6c support the concept that even a single molecule of a highly active proteolytic enzyme may be detected in this assay. The distribution of the mean fluorescent intensities of parallel samples, each incubated with one enzyme molecule per reaction volume (average), followed a Poisson-distribution with a mean (λ) of ˜1 (p>>0.05 in the χ² test performed with the values observed, and those expected for a Poisson-distribution).

In the case of other proteolytic enzymes (e.g., trypsin), the same approach worked well, but the sensitivity was less optimum (data not shown). Biotin- and fluorescent dye-labeled peptides could also be applied in analogous fashion (e.g., in the case of metalloproteinase I), making titration on the appropriate substrate peptide possible; measurement in this case was also convenient, but sensitivity was again less optimal than for proteinase K (data not shown). However, the lower sensitivity in the latter case would still allow the diagnostic application of the assay in a clinical setting.

Flow cytometric measurements were conducted using a Becton-Dickinson FACScan flow cytometer (Mountain View, Calif., USA). Fluorescence signals were collected in the logarithmic mode, and subsequently converted to linear units and the data were analyzed by the BDIS CELLQUEST 3.3 (Becton-Dickinson) software. Samples were run at high speed, the applied laser power was 15 mW, fluorescence signals were detected in the FL1 and FL2 channels, through the 530/30 and 585/42 interference filters of the instrument, respectively.

### Example 5

### Immunoassays Using Biological Microbeads

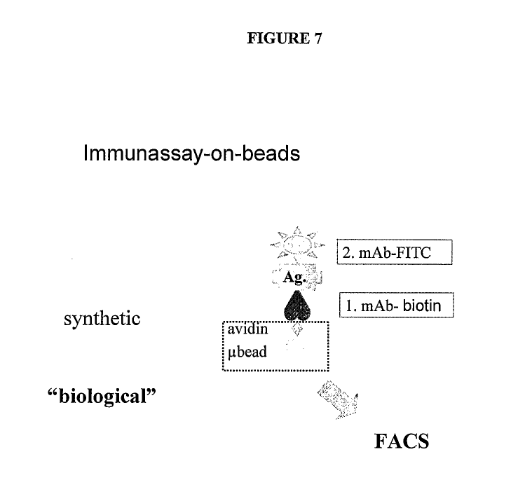

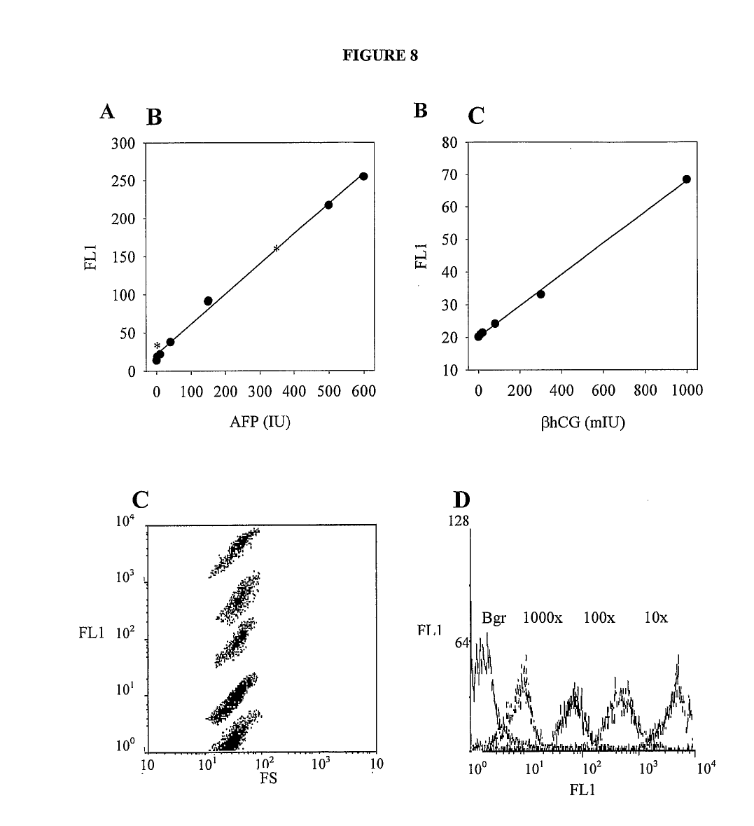

The standard microplate design may be used in accordance with an embodiment of the present invention; specifically, two noncompeting antibodies (i.e., one biotinylated, the other fluorescent) binding to an antigen to be measured to the beads. Using bacteria and yeasts together, the inventors have studied alpha-fetoprotein (“AFP”) and beta human choriogonadotrophin, measured simultaneously in the same tube. The methodology has been shown to yield data that is at least as accurate and reproducible as it is reported for standard microplate procedures. These are illustrated in FIG. 7 and FIG. 8.

Alfa-fetoprotein (AFP) and β-human chorionic gonadotropin hormone (βhCG) were titrated performing the two measurements in the same sample simultaneously. The antibody (Ab) solution contained pairs of noncompeting Abs, biotinylated and fluorescein isothiocyanate-conjugated, respectively, at saturating concentrations. For both AFP and βhCG, the signal and capture MoAbs were from Oy Medix (Finland). “Ab solutions” were prepared by adding 2.5-2.5 μl of signal and capture MoAbs solutions, containing the antibodies at 0.5 and 1 μg/ml concentration, respectively, to 145 μl of 1× PBS. 25 μl of the appropriate “Ab solution” was added to 25 μl aliquots of AFP or βhCG standard solutions prepared by adding small volumes of AFP or βhCG to the same batch of standard maternal serum (from the Isotope Institute, Budapest, Hungary), to minimize matrix effects. Control measurements with international standards for AFP (NIBSC; Isotope Institute, Budapest, Hungary) were also performed. After 30 mins incubation at RT in the dark, 50 μl PBS containing 10,000 biological microbeads was added. The samples were incubated at RT in the dark, for 40 mins, washed and resuspended in 250 μl PBS. The AFP and βhCG samples containing the same serum dilutions were mixed and together analyzed by flow-cytometry.

FIGS. 8a through 8d show a combined determination of AFP and βhCG levels, using appropriate capture and detection antibodies, in combination with avidinated bacteria and yeast cells, respectively, in accordance with an embodiment of the present invention. AFP and βhCG antigens (including international standards shown by asterisks) were captured by biotinylated MoAbs and detected by FITC labeled MoAbs. The regression coefficient for this assay was 0.9994 for AFP, and 0.9987 for βhCG. A titration of a dilution series of AFP are shown in FIG. 8a βhCG in FIG. 8b. FIG. 8c shows a forward-scatter/forward light scattering dot-plot, and FIG. 8d shows a forward light-scattering distribution histogram of a mixture of five yeast cell samples stained with a tenfold dilution series of 1 mg/ml fluorescein isothiocyanate (background fluorescence: Bgr).

The standard curves shown in FIG. 8a have been simultaneously registered, thus AFP and βhCG levels in a particular sample can be measured in a single run. The sensitivity of this immunoassay performed using biological microbeads is similar to what has been described in an analogous bead-assay set up with commercial microbeads (Frengen J. et al., (1995) J Immunol Methods 178:141-51). Thus, biological microbeads can be conveniently used to quantitatively determine antigens in patient samples. The intra-experimental variation is relatively low: the regression coefficients of the titration graphs shown (see legend for FIG. 8a) may satisfy the conditions for routine use.

In the experiment shown in FIG. 8b, the unsynchronized cells of the ND6 yeast strain were addressed to various fluorescence intensities with a single dye (fluorescein isothiocyanate). It is estimated that gating on the oblique subpopulations of the FSC/FL1 dot-plots (FIG. 8c), rather than the FL1 distribution histograms (FIG. 8d), make resolution adequate to distinguish up to 10 subpopulations in a mixed sample. Synchronization of the yeast cultures by α-factor (mating pheromone) has not yielded significantly more uniform size distribution; a mixture of S. cerevisiae, haploid and diploid S. pombe cells, however, was well resolved on the light scatter dot plots (data not shown).

Flow cytometric measurements were conducted using a Becton Dickinson FACScan flow cytometer (Mountain View, Calif., USA). Fluorescence signals were collected in the logarithmic mode, and subsequently converted to linear units and the data were analyzed by the BDIS CELLQUEST 3.3 (Becton-Dickinson) software. Samples were run at high speed, the applied laser power was 15 mW, fluorescence signals were detected in the FL1 and FL2 channels, through the 530/30 and 585/42 interference filters of the instrument, respectively.

While the description above refers to particular embodiments of the present invention, it will be understood that many modifications may be made without departing from the spirit thereof. The presently disclosed embodiments are therefore to be considered in all respects as illustrative and not restrictive.

What is claimed is:

1. A method of binding biological microbeads to a target compound, comprising:
providing a composition comprising a target compound; and
contacting the composition with a quantity of biological microbeads sufficient to bind at least a portion of the target compound to produce a quantity of biological microbead-bound target compounds.

2. The method of claim 1, wherein the biological microbeads comprise proteins that are covalently attached to fixed cells.

3. The method of claim 2, wherein the fixed cells are selected from the group consisting of bacterial cells, yeast cells, and combinations thereof.

4. The method of claim 3, wherein the fixed cells are bacterial cells.

5. The method of claim 3, wherein the bacterial cells comprise Staphylococcus aureus cells.

6. The method of claim 3 wherein the fixed cells comprise yeast cells.

7. The method of claim 3, wherein the yeast cells comprise the strain ND6.

8. The method of claim 2, wherein the proteins are selected from the group consisting of avidin, streptavidin, and combinations thereof.

9. The method of claim 1, wherein the target compound comprises a compound selected from the group consisting of proteins, nucleic acids, antibodies, and combinations thereof.

10. The method of claim 1, wherein the target compound comprises biotin.

11. The method of claim 1, wherein the biological microbead-bound target compounds are detected by flow cytometry.

12. A biological microbead composition, comprising cells that have been fixed and cross-linked to a protein, wherein the protein is adapted to bind a target compound.

13. The composition of claim 12, wherein the fixed cells are selected from the group consisting of bacterial cells, yeast cells, and combinations thereof.

14. The composition of claim 13, wherein the fixed cells comprise bacterial cells.

15. The composition of claim 13, wherein the bacterial cells comprise Staphylococcus aureus.

16. The composition of claim 13, wherein the fixed cells comprise yeast cells.

17. The composition of claim 13, wherein the yeast cells comprise strain ND6.

18. The composition of claim 12, wherein the protein is selected from the group consisting of avidin, streptavidin, and combinations thereof.

19. The composition of claim 12, wherein the target compound comprises a compound selected from the group consisting of proteins, nucleic acids, antibodies, and combinations thereof.

20. The composition of claim 12, wherein the target compound comprises biotin.

21. A method of detecting target compounds, comprising:
providing a composition comprising a target compound;
contacting the composition with a quantity of biological microbeads sufficient to bind at least a portion of the target compound to produce a quantity of biological microbead-bound target compound and a quantity of unbound target compound;
separating the biological microbead-bound target compound from the unbound target compound; and
detecting the biological microbead-bound target compound.

22. The method of claim 19, wherein substantially all of the target compound is bound by the biological microbeads.
